(19) United States
(12) Patent Application Publication
(10) Pub. No.: US 2016/0375231 A1
(43) Pub. Date: Dec. 29, 2016

(54) Fecal Matter Transplant Kit and Method of Using Same

(71) Applicant: Operating Room Innovations, Incorporated, Coronado, CA (US)

(72) Inventors: Ruel Flores Salvador, San Diego, CA (US); Phong Duy Bui, San Diego, CA (US)

(73) Assignee: Operating Room Innovations, Incorporated, Coronado, CA (US)

(21) Appl. No.: 15/263,457

(22) Filed: Sep. 13, 2016

Related U.S. Application Data
(62) Division of application No. 14/142,866, filed on Dec. 29, 2013.

(51) Int. Cl. A61M 31/00; A61B 1/31; A61J 15/00; A61B 10/00

(52) U.S. Cl. CPC A61M 31/00; A61B 10/0038; A61B 1/31; A61J 15/0003; A61M 2202/068; A61M 2205/7545

(57) ABSTRACT

A method for transplanting a fecal matter/saline solution suspension within a patient, wherein the method uses a transplant kit that includes a fecal matter storage container having a fecal matter filter, a fecal matter/saline container connected to the fecal matter storage container for mixing the fecal matter and the saline to create a suspension, and a suspension delivery device for transplanting the suspension into the patient.

### FIELD OF THE INVENTION

This invention relates generally to fecal matter transplant kits and more particularly to a fecal matter transplant kit that includes a fecal matter storage container having a fecal matter filter, a fecal matter/saline container connected to the fecal matter storage container for mixing the fecal matter and the saline to create a suspension and a suspension delivery device for transplanting the suspension into the patient.

### BACKGROUND OF THE INVENTION

Prior to the present invention, as set forth in general terms above and more specifically below, it is known, to treat gastro-intestinal disorders such as Clostridium difficile, often called C. difficile or C. diff, through bacteriotherapy such as antibiotics, probiotics or fluids. See for example, U.S. Pat. No. 8,197,805 to Lin et al., U.S. Pat. No. 8,110,177 to Lin et al., U.S. Pat. No. 6,645,530 to Borody, U.S. Pat. No. 6,635,260 to Gerding, U.S. Pat. No. 5,443,826 to Borody, U.S. Patent Application No. 2013/0045274 to Hlavka, U.S. Pat. No. 8,371,306 to Haines, et al., U.S. Pat. No. 8,322,345 to West, U.S. Pat. No. 8,100,130 to Allen, et al., U.S. Pat. No. 7,958,894 to Katoh, et al., U.S. Pat. No. 7,114,500 to Bonutti, U.S. Pat. No. 6,843,252 to Harrison, et al., U.S. patent, and The Therapeutic Advances in Gastroenterology to Rohlke et al., which was published in 2012, and relates to Fecal Microbiota Transplantation in Relapsing Clostridium difficile Infection. While the use of bacteriotherapy to treat gastro-intestinal disorders such as Clostridium difficile may have been generally satisfactory, there is nevertheless a need for a fecal matter transplant kit that is safe and efficient to use and that includes a standardized system to collect and transplant fecal matter from one individual to another individual.

It is a purpose of this invention to fulfill this and other needs in the art in a manner more apparent to the skilled artisan once given the following disclosure.

### SUMMARY OF THE INVENTION

A feature of the present invention is a fecal matter transplant kit that includes a fecal matter storage container having a fecal matter filter, a fecal matter/saline container connected to the fecal matter storage container for mixing the fecal matter and the saline to create a suspension, and a suspension delivery device for transplanting the suspension into the patient.

Another feature of the present invention is the provision of a fecal matter storage container including a sealable bag.

Another feature of the present invention is the provision of a medical grade polymeric sealable bag.

Another feature of the present invention is the provision of a fecal matter storage container having a sealable opening located at one end of the fecal matter storage container.

Another feature of the present invention is the provision of a fecal matter storage container including a fluid flow port located at the other end of the fecal matter storage container.

Another feature of the present invention is the provision of a fecal matter storage container having a fluid flow shut off means located substantially adjacent to the fluid flow port.

Another feature of the present invention is the provision of a fecal matter filter including a net filter.

Another feature of the present invention is the provision of a fecal matter/saline container including a fecal matter/saline bag.

Another feature of the present invention is the provision of a medical grade polymeric fecal matter/saline bag.

Another feature of the present invention is the provision of a fecal matter/saline container having a fluid flow port located at one end of the fecal matter/saline container.

Another feature of the present invention is the provision of a fecal matter/saline container including a fluid flow shut off means located substantially adjacent to the fluid flow port.

Another feature of the present invention is the provision of a fecal matter/saline container having a sterile solution port located substantially adjacent to the fluid flow port.

Another feature of the present invention is the provision of a suspension delivery device having a fecal matter and saline suspension fluid conduit.

Another feature of the present invention is the provision of a fecal matter and saline suspension fluid conduit including polymeric tubing.

Another feature of the present invention is the provision of a suspension delivery device having a fecal matter/saline container connector located at one end of the conduit.

Another feature of the present invention is the provision of a suspension delivery device including a colonoscope or nasogastric tube connector located at another end of the conduit.

A further feature of the present invention is the provision of a method for transplanting a fecal matter/saline suspension within a patient, wherein the method includes the steps of: collecting a specimen of fecal matter; determining if the specimen is healthy enough to be transplanted into the patient; depositing the specimen into a fecal matter storage container; sealing the fecal matter storage container; introducing an amount of a sterile solution into the fecal matter storage container; mixing the fecal matter specimen and the amount of sterile solution in the fecal matter storage container to create a fecal matter and sterile solution mixture; transferring the fecal matter and sterile solution mixture into a fecal matter/saline container; mixing the fecal matter and sterile solution mixture with another amount of the sterile solution in the fecal matter/saline container to create a fecal matter and sterile solution suspension; and using a suspension delivery device to transplant the fecal matter/saline solution suspension within the patient.

Another feature of the present invention is the provision of a saline sterile solution.

Another feature of the present invention is the provision of a method for transplanting a fecal matter/saline suspension within a patient, wherein the step of using a suspension delivery device further includes the steps of: using a fluid pump to withdraw the fecal matter/saline solution suspension from the fecal matter/saline container and to transfer the fecal matter/saline solution suspension to a colonoscope or nasogastric tube; and using the colonoscope or nasogastric tube to deliver the fecal matter/saline solution suspension into the patient.

A still further feature of the present invention is the provision of a fecal matter transplant kit including: a fecal matter storage container having a fecal matter filter; a fecal matter/saline container operatively connected to the fecal matter storage container for mixing the fecal matter and the saline to create a suspension; and a suspension delivery device for transplanting the suspension into the patient, wherein the suspension delivery device includes a colonoscope.

The preferred fecal matter transplant kit, according to various embodiments of the present invention, offers the following advantages: ease of use, economy of design, transplant personnel do not have to contact the fecal matter, fecal matter odor is substantially eliminated, the need to blend the fecal matter suspension is eliminated, and a safe, standardized system to collect and transplant the fecal matter is utilized.

### BRIEF DESCRIPTION OF DRAWINGS

The above mentioned features and steps of the invention and the manner of attaining them will become apparent, and the invention itself will be best understood by reference to the following description of the embodiments of the invention in conjunction with the accompanying drawings, wherein like characters represent like parts throughout the several views and in which:

FIG. 1 is a schematic illustration of a fecal matter transplant kit, constructed according to the present invention;

FIG. 2 is a schematic illustration of a fecal matter storage container of the fecal matter transplant kit, constructed according to the present invention;

FIG. 3 is a schematic illustration of a fecal matter/saline container of the fecal matter transplant kit, constructed according to the present invention;

FIG. 4 is a schematic illustration of a fecal matter/saline suspension delivery device of the fecal matter transplant kit, constructed according to the present invention; and

FIG. 5 is a flowchart of a method of using the fecal matter transplant kit, according to the present invention.

### DETAILED DESCRIPTION OF PREFERRED EMBODIMENTS

To provide some perspective that will be helpful in understanding and appreciating the inventive concepts of the present invention, it is well known that Clostridium difficile (C. Diff.) causes severe diarrhea and other intestinal diseases. Interestingly, it is not uncommon for individuals to accidentally ingest spores of Clostridium difficile while they are patients in a hospital or other care giving facility including long term care facilities. In fact, over 14,000 people a year in the United States alone die as a result. This is because as the bacteria overgrow, they release toxins that attack the lining of the intestines. If the normal gut flora in the patient has been destroyed through the use of antibiotics, the person's digestive system becomes devoid of the bacteria needed to fight off these harmful toxins. If not properly treated, these toxins can become life threatening. The present invention can be used to treat Clostridium difficile by transplanting a healthy stool specimen within a patient affected with Clostridium difficile such that the patient's digestive system is infiltrated with the bacteria needed to fight off these harmful toxins.

As is known in past bacteriotherapy treatments for Clostridium difficile, the medical personnel may inadvertently come into contact with fecal matter. Also, prior, known bacteriotherapy treatments for Clostridium difficile do not necessarily use sealed containers which can result in a fecal matter odor being noticed by the medical personnel and the patient. Also, previous bacteriotherapy treatments for Clostridium difficile require a blending or mechanical mixing of the fecal matter and the saline solution which then requires costly sanitation procedures to properly clean the mixing equipment. Therefore, there is clearly a need for a safe, standardized system to collect and transplant the fecal matter.

The present invention addresses the need for a safe, standardized system to collect and transplant the fecal matter by allowing the medical personnel to create a fecal matter and saline suspension without having to physically contact the fecal matter. Also, once the fecal matter has been placed within the fecal matter transplant kit 2, the fecal matter odor should be substantially reduced because the fecal matter is now located within sealed containers. Finally, the present invention does not require that the medical professional to blend or otherwise mechanically mix the fecal matter with the saline solution. This elimination of the blending step significantly reduces the clean-up time and possible likelihood of the medical personnel being contaminated by fecal matter.

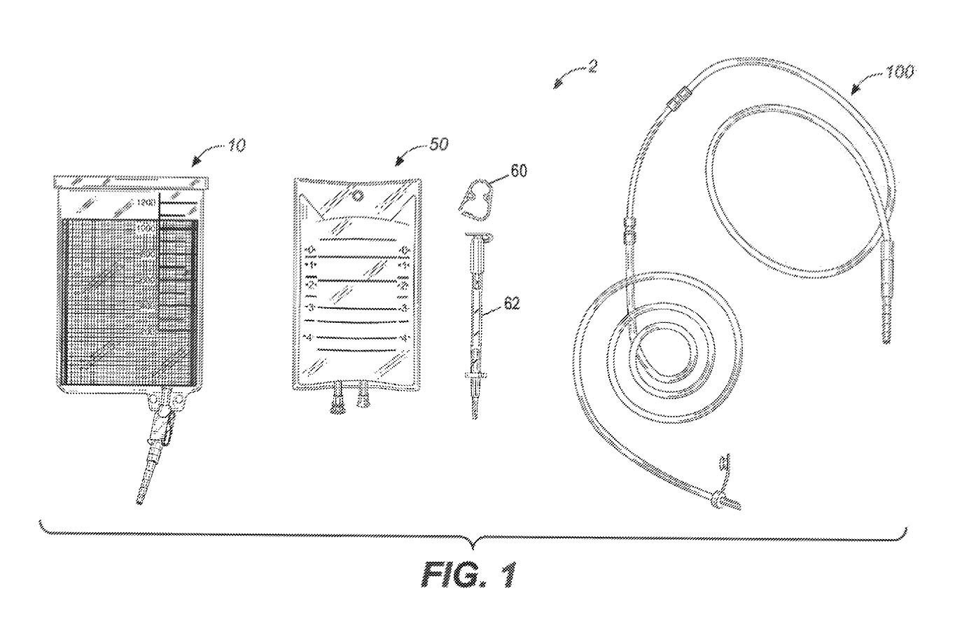

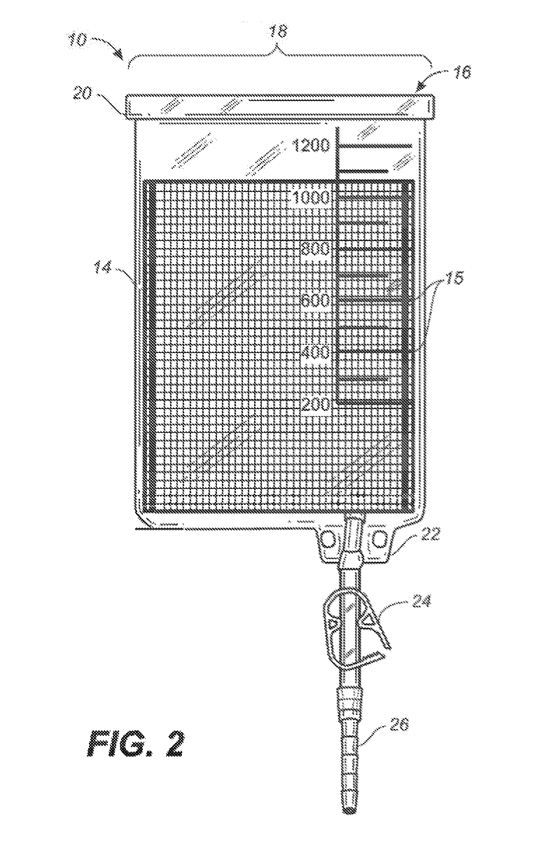

Considering now the fecal matter transplant kit 2 in greater detail with reference to FIGS. 1-2, the fecal matter kit 2 generally includes a fecal matter storage container assembly 10 having a fecal matter filter 14, a fecal matter/saline container assembly 50 operatively connected to the fecal matter storage container assembly 10 for mixing the fecal matter and the saline to create a suspension, and a suspension delivery device 100 for transplanting the suspension into the patient. As will be described in greater detail later, fecal matter storage container assembly 10 is used to mix and filter the fecal matter and saline, fecal matter/saline container assembly 50 is used to first store the saline solution and then to store the fecal matter and saline suspension, and the suspension delivery device 100 is used for transplanting the suspension into the patient.

Considering now the container or bag assembly 10, the container assembly 10 generally comprises a closable container or bag 20 which includes a net filter 14 located substantially within bag 20 and a shut off port 22. As more clearly shown in FIG. 2, bag 20, preferably, is constructed of any suitable medical grade polymeric material such as polyvinyl chloride (PVC) or other suitable PVC substitute. Located at one end of bag 20 is an opening 16 wherein fecal matter can be introduced into bag 20 by conventional techniques. After the fecal matter is introduced into bag 20, opening 16 is then conventionally closed or sealed along the length 18 of bag 20 in order to retain the fecal matter substantially within bag 20. Located substantially within bag 20 is net filter 14. Preferably, net filter 14 is constructed of any suitable medical grade material such as polypropylene or other suitable polymer materials that has a mesh size range such that net filter 14 is capable of filtering solid particles of the stool.

Also, net filter 14 is attached to the interior of bag 20 by conventional techniques such as thermal welding or other similar techniques. Volume markings 15 are utilized to determine the amount of fecal matter and sterile solution within bag 20. Located at the other end of bag 20 is shut off port 22. A conventional shut off valve or pinch clamp 24 is located adjacent to shut of port 22 in order to keep any fecal matter from leaving bag 20 while the fecal matter is being placed within bag 20. Pinch clamp 24, preferably, is constructed of polypropylene. A connector 26 is attached to port 22 so that connector 26 interacts with a saline bag port 54 formed in the fecal matter/saline container assembly 50, as will be described in greater detail later. Connector 26, preferably, is constructed of polypropylene.

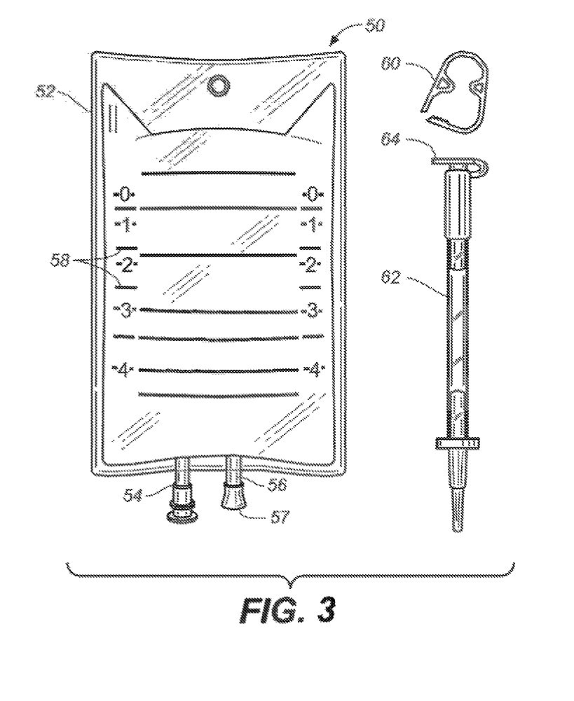

Regarding FIG. 3, saline bag or container assembly 50 is illustrated. Saline bag or container assembly 50 further includes bag 52, ports openings 54, 56, port cap 57 and indicia volume markings 58. Preferably, bag 52 is constructed of any suitable medical grade polymeric material, such as PVC. Bag 52, preferably, is constructed so as to hold 500 ml of saline but different size bags can also be used. Located at one end of bag 52 is the port opening 54 which is used to interact with container or bag assembly 10, as will be described in greater detail later. Port opening 56 is utilized to introduce a suitable saline or other suitable sterile solution into bag 52. Port cap 57 is placed over port 56 after the saline solution is placed within bag 52. Volume markings 58 are utilized to determine the amount of sterile solution within bag 52. A conventional shut off valve or pinch clamp 60 is located adjacent to shut off port 54 is order to keep any fecal matter from leaving fecal matter/saline container bag 52 after the fecal matter is being placed within bag 52. Pinch clamp 60, preferably, is constructed of polypropylene. A connector 62 is operatively attached port 54 and to suspension delivery assembly 100 so that connector 62 interacts with port 54 in order to transport the fecal matter/saline solution suspension to suspension delivery assembly 100, as will be described in greater detail later. Connector 62, preferably, is constructed of PVC. Connector cap 64 is placed over connector 62 in order to keep any contaminants from entering connector 62 prior to its use.

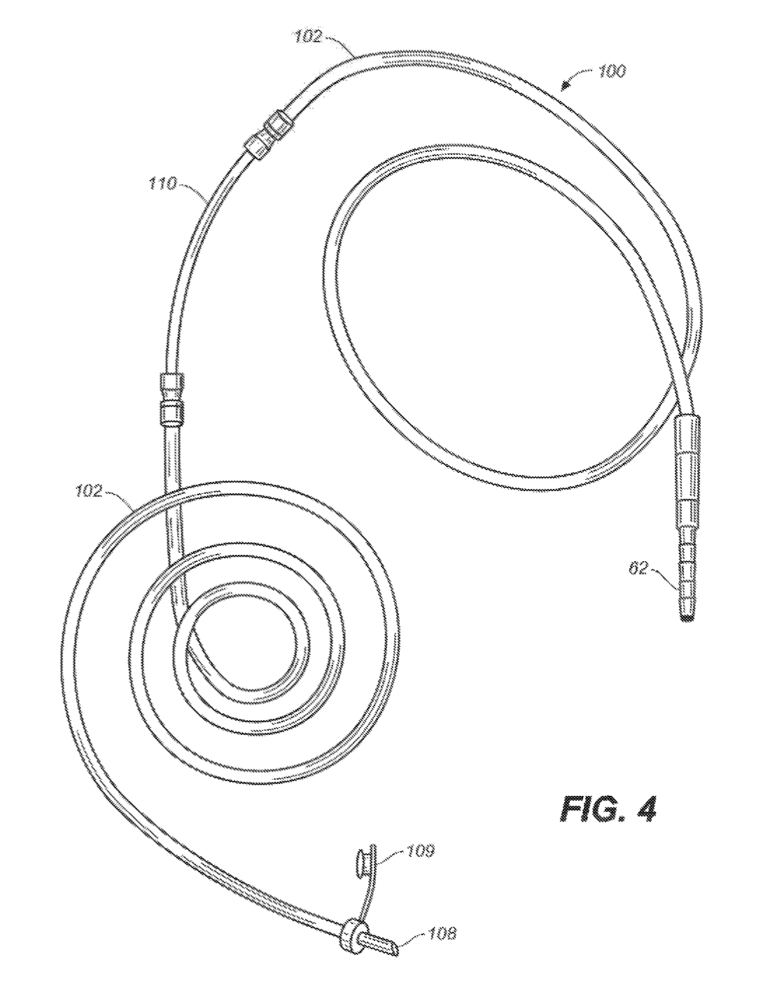

With respect to FIG. 4, the fecal matter/sterile solution suspension delivery assembly 100 is illustrated. Suspension delivery assembly 100 includes, in part, tubing 102, saline bag or container assembly connector 62, colonoscope injector port connector 108, colonoscope injector port connector cap 109, and fluid pump connection tubing 110. Preferably, tubing 102 and 110 are constructed of any suitable medical grade polymeric material such as PVC or the like and should be made as long as conventional fluid pump tubing. Located at one end of tubing 102 is a polymeric connector 62 that allows tubing 102 to be conventionally connected to saline bag or container assembly 50 at port 54, as described earlier. Located at the other end of tubing 102 is a polymeric male luer lock connector 108 that allows tubing 102 to be conventionally connected to a colonoscope or nasogastric tube injector port (not shown). A conventional colonoscope injector port connector cap 109 is located adjacent to connector 108 in order to keep contaminants from entering connector 108. Located in between connectors 62 and 108 is tubing 110 which is used to connect suspension delivery assembly 100 to a conventional fluid pump. Under the present invention, tubing 102 is used to transport the fecal matter/sterile solution suspension from the saline bag or container assembly 50 to the colonoscope injector port, as will be described in greater detail later.

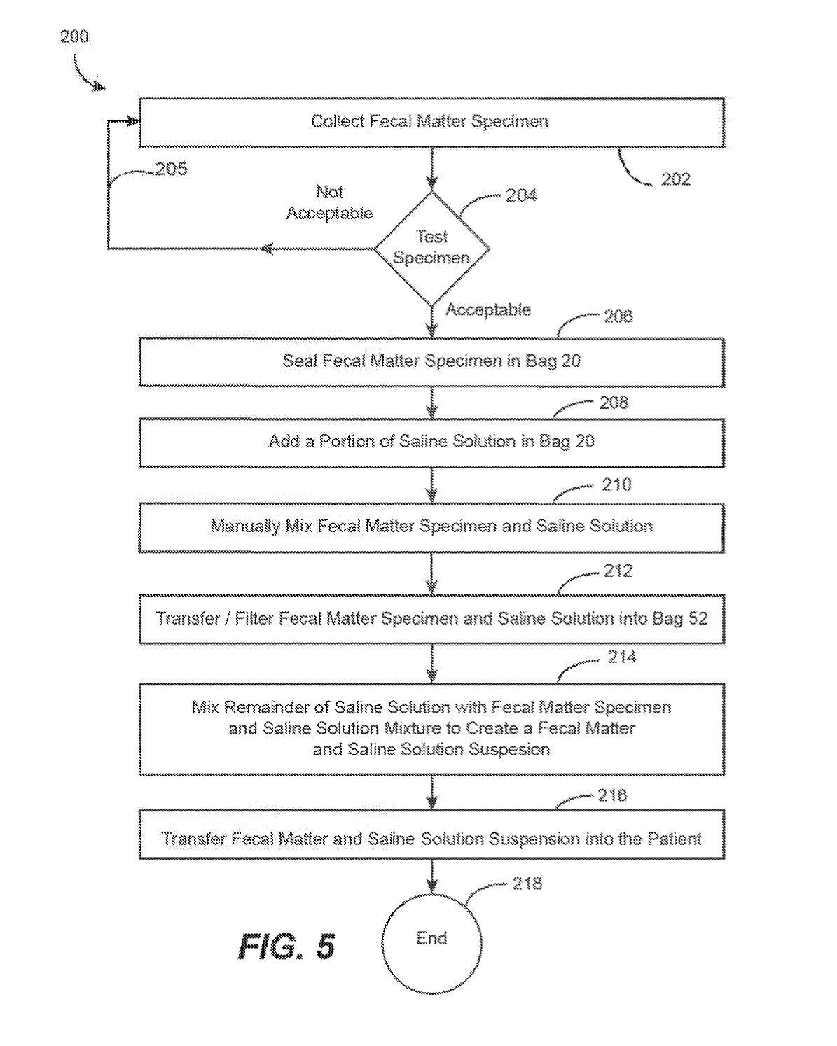

With reference to FIG. 5, the method 200 of operating fecal matter transplant kit 2 is described. During the operation of fecal matter transplant kit 2, preferably, a fecal matter specimen of two (2) ounces or less is collected from the patient's relatives and/or friends on the morning of the procedure (step 202). The donor is instructed to put the specimen in a conventional, medical grade specimen cup for testing to determine if the specimen is disease free. Once it has been determined that the specimen is acceptable (step 204), the specimen is then placed in the container or bag assembly 10 (FIG. 2) and bag 20 is sealed along location 18 in order to retain the fecal matter specimen within bag 20. If the specimen is determined not to be acceptable, then a new specimen must be harvested (step 205).

Once the fecal matter specimen has been sealed within bag 20 (step 206), bag 20 is connected to saline bag or container assembly 50 by attaching port 22 on bag 20 to port 54 on saline bag 52 (FIG. 3) through connector 26 (step 208). After a predetermined amount of saline or sterile solution has been introduced from saline bag or container assembly 50 into bag 20, shut off valve or pinch clamp 24 (FIG. 2) is activated to keep further sterile solution from entering bag 20 through connector 26. It is to be understood that clamp 24 does not have to be activated so that all the saline from bag 52 can go in and out of bag 20. Thereafter, the fecal matter specimen and the sterile solution located within bag 20 are manually mashed up in order to pulverize any big chunks in the fecal matter specimen (step 210). It is to be understood that the specimen is manually mashed until there are no more big chunks of stool.

After the fecal matter specimen and the sterile solution have been mashed up, shut off valve 24 is opened so that the pulverized fecal matter specimen and the sterile solution is transferred from the bag 20 into saline bag or container assembly 50 through connector 26. During this step, net filter 14 (FIG. 2) is used to filter out any remaining large chunks of fecal matter and/or undigested food particulates (step 212). After the fecal matter specimen and the sterile solution have been transferred into saline bag or container assembly 50, bag 20 is then properly disposed of.

Once the fecal matter specimen and the sterile solution have been completely transferred into saline bag or container assembly 50, shut off valve 60 (FIG. 3) is activated in order to keep all of the fecal matter specimen and the sterile solution within bag 52. After the fecal matter specimen and the sterile solution have been placed within bag 52, the fecal matter specimen and the sterile solution maybe further manually mixed within bag 52 to create a fecal matter and sterile solution suspension (step 214), if needed.

In order to transport the fecal matter and sterile solution suspension from bag 52 into the patient, suspension delivery assembly 100 is utilized (step 216). More precisely, connector 62 on tubing 102 (FIG. 4) is attached to saline bag 52 at port 54. Connector 108 is connected to the injection port of colonoscope (not shown). Fluid pump tubing 110 is conventionally connected to a conventional fluid pump. After the various connections are made between bag 52 and the fluid pump and the colonoscope or nasogastric tube, the medical personnel use the fluid pump to withdraw the fecal matter and sterile solution suspension from bag 52 and force the fecal matter and sterile solution suspension through the injection port of the colonoscope or nasogastric tube. The healthy fecal matter and sterile solution suspension is then conventionally sprayed throughout the colon. It is to be understood that there is no desired flow rate. The important thing is to introduce healthy bacteria.

The preceding merely illustrates the principles of the invention. It will thus be appreciated that those skilled in the art will be able to devise various arrangements which, although not explicitly described or shown herein, embody the principles of the invention and are included within its spirit and scope. Furthermore, all examples and conditional language recited herein are principally intended expressly to be only for pedagogical purposes and to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

This description of the exemplary embodiments is intended to be read in connection with the figures of the accompanying drawing, which are to be considered part of the entire written description. In the description, relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents to the extent such incorporated materials and information are not inconsistent with the description herein.

The written description portion of this patent includes all claims. Furthermore, all claims, including all original claims as well as all claims from any and all priority documents, are hereby incorporated by reference in their entirety into the written description portion of the specification, and Applicant(s) reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended and those skilled in the art will recognize, that the invention embraces and is also thereby described in terms of any individual member or subgroup of members of the Markush group.

Other embodiments are within the following claims. Therefore, the patent may not be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

Other modifications and implementations will occur to those skilled in the art without departing from the spirit and the scope of the invention as claimed. Accordingly, the description hereinabove is not intended to limit the invention, except as indicated in the appended claims.

1. A method for transplanting a fecal matter/saline solution suspension within a patient, wherein the method is further comprised of the steps of:
collecting a specimen of fecal matter;
determining if the specimen is healthy enough to be transplanted into the patient;
depositing the specimen into a closable fecal matter storage container assembly having a sealable opening at a receiving end thereof;
sealing the closable fecal matter storage container assembly;
introducing a first amount of a sterile solution from a fecal matter/saline container assembly into the closable fecal matter storage container assembly;
mixing the fecal matter specimen and the first amount of sterile solution in the closable fecal matter storage container assembly to create a fecal matter and sterile solution mixture;
transferring the fecal matter and sterile solution mixture from the closable fecal matter storage container assembly into the fecal matter/saline container assembly;
mixing the fecal matter and sterile solution mixture with a second amount of sterile solution in the fecal matter/saline container assembly to create a fecal matter/sterile solution suspension; and
using a suspension delivery device to transplant the fecal matter/saline solution suspension within the patient.

2. The method for transplanting a fecal matter/saline solution suspension within a patient, as in claim 1, wherein the sealing step is further comprised of the step of:
sealing the receiving end of the closable fecal matter storage container assembly in order to retain the fecal matter specimen and the first amount of a sterile solution within the closable fecal matter storage container assembly.

3. The method for transplanting a fecal matter/saline solution suspension within a patient, as in claim 1, wherein the introducing step is further comprised of the step of:
introducing the first amount of the sterile solution from the fecal matter/saline container assembly into the closable fecal matter storage container assembly through the use of a shut-off port located at a discharge end of the closable fecal matter storage container assembly and a filtered fecal matter port opening attached to the fecal matter/saline container assembly.

4. The method for transplanting a fecal matter/saline solution suspension within a patient, as in claim 3, wherein the step of mixing of the fecal matter specimen and the first amount of sterile solution in the closable fecal matter storage container assembly is further comprised of the step of:
using a fecal matter filter attached to an interior of the closable fecal matter storage assembly in order to filter solid fecal matter particles from the fecal matter received through the opening, wherein the fecal matter filter has a sufficiently small mesh size for filtering solid fecal matter particles to facilitate separating undigested food particulates and large chucks of fecal matter from the fecal matter introduced through the sealable opening into the closable fecal matter storage assembly.

5. The method for transplanting a fecal matter/saline solution suspension within a patient, as in claim 4, wherein the transferring step is further comprised of the step of:
transferring the filtered fecal matter and sterile solution mixture from the closable fecal matter storage container assembly into the fecal matter/saline container assembly through the use of the shut-off port located at the discharge end of the closable fecal matter storage container assembly and the filtered fecal matter port opening attached to the fecal matter/saline container assembly.

6. The method for transplanting a fecal matter/saline solution suspension within a patient, as in claim 5, wherein the step of mixing the fecal matter and sterile solution mixture with the second amount of sterile solution in the fecal matter/saline container assembly is comprised of the steps of:
activating a shut off valve attached to the fecal matter/saline container assembly in order to keep all of the fecal matter and sterile solution mixture within the fecal matter/saline container assembly; and
manually mixing the fecal matter and sterile solution mixture with the second amount of sterile solution in the fecal matter/saline container assembly to create the fecal matter and sterile solution suspension.

7. The method for transplanting a fecal matter/saline solution suspension within a patient, as in claim 6, wherein the step of using a suspension delivery device is further comprised of the steps of:
using a fluid pump to withdraw the fecal matter/saline solution suspension from the fecal matter/saline container assembly through the use of a port located on the fecal matter/saline container assembly;
transferring the fecal matter/saline solution suspension through the port located on the fecal matter/saline container assembly to a colonoscope or nasogastric tube; and
using the colonoscope or nasogastric tube to deliver the fecal matter/saline solution suspension into the patient.

8. The method for transplanting a fecal matter/saline solution suspension within a patient, as in claim 1, wherein the sterile solution is a saline solution.

9. A method of using a fecal matter transplant kit, wherein the method is further comprised of the steps of:
depositing an amount of fecal matter into a closable fecal matter storage bag having a closable opening at a first end thereof;
closing the closable fecal matter storage bag;
introducing a first amount of a sterile solution from a fecal matter/saline bag into the closable fecal matter storage bag;
manually mixing the fecal matter specimen and the first amount of sterile solution in the closable fecal matter storage bag to create a fecal matter and sterile solution mixture;
transferring the fecal matter and sterile solution mixture from the closable fecal matter storage bag into the fecal matter/saline bag;
manually mixing the fecal matter and sterile solution mixture with a second amount of sterile solution in the fecal matter/saline bag to create a fecal matter and sterile solution suspension; and
using a suspension delivery device to transplant the fecal matter/saline solution suspension within a patient.

10. The method for using a fecal matter transplant kit, as in claim 9, wherein the closing step is further comprised of the step of:
closing the first end of the closable fecal matter storage bag in order to retain the fecal matter specimen and the first amount of a sterile solution within the closable fecal matter storage bag.

11. The method for using a fecal matter transplant kit, as in claim 9, wherein the introducing step is further comprised of the step of:
introducing the first amount of the sterile solution from the fecal matter/saline bag into the closable fecal matter storage bag through the use of a shut-off port located at a second end of the closable fecal matter storage bag and a filtered fecal matter port opening attached to the fecal matter/saline bag.

12. The method for using a fecal matter transplant kit, as in claim 11, wherein the step of mixing of the fecal matter specimen and the first amount of sterile solution in the closable fecal matter storage bag is further comprised of the step of:
using a fecal matter filter attached to an interior of the closable fecal matter storage bag in order to filter solid fecal matter particles from the fecal matter received through the opening, wherein the fecal matter filter has a sufficiently small mesh size for filtering solid fecal matter particles to facilitate separating undigested food particulates and large chucks of fecal matter from the fecal matter introduced through the closable opening into the closable fecal matter storage bag.

13. The method for using a fecal matter transplant kit, as in claim 12, wherein the transferring step is further comprised of the step of:
transferring the filtered fecal matter and sterile solution mixture from the closable fecal matter storage bag into the fecal matter/saline bag through the use of the shut-off port located at the second end of the closable fecal matter storage bag and the filtered fecal matter port opening attached to the fecal matter/saline bag.

14. The method for using a fecal matter transplant kit, as in claim 13, wherein the step of mixing the fecal matter and sterile solution mixture with the second amount of sterile solution in the fecal matter/saline bag is comprised of the steps of:
activating a shut off valve attached to the fecal matter/saline bag in order to keep all of the fecal matter and sterile solution mixture within the fecal matter/saline bag; and
manually mixing the fecal matter and sterile solution mixture with the second amount of sterile solution in the fecal matter/saline bag to create the fecal matter and sterile solution suspension.

15. The method for using a fecal matter transplant kit, as in claim 14, wherein the step of using a suspension delivery device is further comprised of the steps of:
using a fluid pump to withdraw the fecal matter/saline solution suspension from the fecal matter/saline bag through the use of a port located on the fecal matter/saline bag;
transferring the fecal matter/saline solution suspension through the port located on the fecal matter/saline bag to a colonoscope or nasogastric tube; and
using the colonoscope or nasogastric tube to deliver the fecal matter/saline solution suspension into the patient.

16. The method for using a fecal matter transplant kit, as in claim 9, wherein the sterile solution is a saline solution.

17. A method for transplanting a fecal matter/saline solution suspension within a patient through the use of a fecal matter transplant kit, wherein the method is further comprised of the steps of:
depositing a specimen of fecal matter into a closable fecal matter storage container assembly having a sealable opening at a receiving end thereof;
sealing the closable fecal matter storage container assembly;
introducing a first amount of a sterile solution from a fecal matter/saline container assembly into the closable fecal matter storage container assembly;
manually mixing the fecal matter specimen and the first amount of sterile solution in the closable fecal matter storage container assembly to create a fecal matter and sterile solution mixture;
transferring the fecal matter and sterile solution mixture from the closable fecal matter storage container assembly into the fecal matter/saline container assembly;
mixing the fecal matter and sterile solution mixture with a second amount of sterile solution in the fecal matter/saline container assembly to create a fecal matter/sterile solution suspension; and
using a suspension delivery device to transplant the fecal matter/saline solution suspension within the patient.

18. The method for transplanting a fecal matter/saline solution suspension within a patient through the use of a fecal matter transplant kit, as in claim 17, wherein the introducing step is further comprised of the step of:
introducing the first amount of the sterile solution from the fecal matter/saline container assembly into the closable fecal matter storage container assembly through the use of a shut-off port located at a discharge end of the closable fecal matter storage container assembly and a filtered fecal matter port opening attached to the fecal matter/saline container assembly.

19. The method for transplanting a fecal matter/saline solution suspension within a patient, as in claim 18, wherein the step of mixing of the fecal matter specimen and the first amount of sterile solution in the closable fecal matter storage container assembly is further comprised of the step of:
using a fecal matter filter attached to an interior of the closable fecal matter storage assembly in order to filter solid fecal matter particles from the fecal matter received through the opening, wherein the fecal matter filter has a sufficiently small mesh size for filtering solid fecal matter particles to facilitate separating undigested food particulates and large chucks of fecal matter from the fecal matter introduced through the sealable opening into the closable fecal matter storage assembly.

20. The method for transplanting a fecal matter/saline solution suspension within a patient, as in claim 19, wherein the step of mixing the fecal matter and sterile solution mixture with the second amount of sterile solution in the fecal matter/saline container assembly is comprised of the steps of:
activating a shut off valve attached to the fecal matter/saline container assembly in order to keep all of the fecal matter and sterile solution mixture within the fecal matter/saline container assembly; and
manually mixing the fecal matter and sterile solution mixture with the second amount of sterile solution in the fecal matter/saline container assembly to create the fecal matter and sterile solution suspension.
